(19) United States
(12) Patent Application Publication
(10) Pub. No.: US 2016/0000374 A1
(43) Pub. Date: Jan. 7, 2016

(54) SMART KNITTED FABRICS

(71) Applicant: DREXEL UNIVERSITY, Philadelphia, PA (US)

(72) Inventors: Kapil R. Dandekar, Philadelphia, PA (US); Genevieve Dion, Philadelphia, PA (US); Adam K. Fontecchio, Dowingtown, PA (US); Timothy P. Kurzweg, Philadelphia, PA (US); Damiano Patron, Philadelphia, PA (US); Owen Montgomery, Philadelphia, PA (US)

(21) Appl. No.: 14/769,478

(22) PCT Filed: Mar. 5, 2014
(86) PCT No.: PCT/US14/20681

§ 371 Date: Aug. 21, 2015

Related U.S. Application Data
(60) Provisional application No. 61/772,670, filed on Mar. 5, 2013.
(60) Provisional application No. 61/906,883, filed on Nov. 20, 2013.

(51) Int. Cl. A61B 5/00; A61B 5/0205; D04B 35/00; A61B 5/0476; D04B 1/12; D04B 1/24

(52) U.S. Cl. CPC A61B 5/6804; D04B 1/12; D04B 1/24; D04B 35/00; A61B 5/4362; A61B 5/4356; A61B 5/4818; A61B 5/4519; A61B 5/0476; A61B 5/0205; A61B 5/0024; A61B 5/742; A61B 2019/448

(57) ABSTRACT

Knitting machines are used to intermesh conductive yarns into loops resulting in knitted fabrics. The knitting machine is adapted to import different types of yarns (conductive and non-conductive) directly into the knit structure. Combining conductive yarns and knitting systems allows for integration of electrical or mechanical component designs into existing clothing fabrication processes, avoiding current limitations of attaching or gluing conductive fabrics or other components over various materials. Starting with a planar design of an antenna, RFID tag, or some other electronic structure, the layout is converted into a CAD knitting program including a grid representing stitches. The CAD specifications of the final design/product are exported to the knitting machines so that the knitting machine may make conductive fabrics in accordance with the CAD specifications. The conductive fabrics are knitted into a variety of garments that monitor the vital signs of the wearer, including the fetus of an expectant mother.

### CROSS REFERENCE TO RELATED APPLICATIONS

This application claims benefit of Provisional Application No. 61/772,670 filed Mar. 5, 2013, and of Provisional Application No. 61/906,883 filed Nov. 20, 2013. The contents of these patent applications are hereby incorporated by reference in their entireties.

### TECHNICAL FIELD

The invention relates to smart knitted fabrics and structures made from conductive yarns and the use of such fabrics in garments to create electrical and mechanical structures. The invention also relates to garments including such structures for use in a wide variety of sensing, communication, and tactile interaction applications.

### BACKGROUND

Numerous researchers have attempted to develop conductive threads and so-called “smart fabrics.” Devices including garments made from smart fabrics are expected to make an enormous contribution to health care by increasing patient safety and comfort and replacing bulky medical instrumentation to measure patient data. These types of devices are also expected to be use in a wide variety of sensing, communication, and tactile interaction applications. In conventional smart fabrics, sensors are being widely studied and used as an essential component, especially in the fields of medical and athletic applications. Such wearable sensors provide a means to monitor the wearer's health through physiological measurements in a natural setting or can be used to detect or alert care providers to potential hazards around the wearer. However, to date, such garments have generally incorporated sewn-in sensors and generally have not been adaptable enough in design to create the variety of shapes and sizes needed for most useful applications.

More recently, integration of wireless smart devices into clothing has received considerable attention. The feasibility of building electrical devices using conductive fabrics has been analyzed through electrical characterization of textile transmission lines, and mounted wearable transmission lines and antennas where conductive fabrics have been applied onto woven fabrics have been demonstrated by, for example, D. L. Paul et al. in “Textile Broadband E-Patch Antenna at ISM Band,” IET Seminar on Antennas and Propagation for Body-Centric Wireless Communications, 2007. Previous work shows conductive copper foils or fabrics bonded to a flexible substrate. However, these techniques show limitations in terms of electrical losses caused by adhesives or glue chemicals. It is desirable to address these drawbacks by knitting conductive and non-conductive yarns in a single process resulting in smart textiles that are unobtrusively integrated into the host garment so as to eliminate the need for chemical adhesives that degrade electrical performance.

Merilampi et al. in “Printed passive UHF RFID tags as wearable strain sensors,” 3^{rd} International Symposium on Applied Sciences in Biomedical and Communication Technologies (ISABEL), 2010, propose the design of a strain-sensor tag fabricated over a stretchable substrate, where the deformation of the conductive layout causes a change of the overall conductivity. One of the most innovative techniques for designing strain sensors is the use of resonant antennas. Previously, it has been demonstrated that a radiating element can be applied for strain and crack monitoring. Mechanical changes of an object are monitored through elongation or relaxation of the antenna size. This change in shape produces a down or up shift of the antenna's resonant frequency, as noted by Cook et al. in “Passive low-cost inkjet-printed smart skin sensor for structural health monitoring,” IET Microwaves, Antennas & Propagation, Vol. 6, N. 14, 2012.

The inventors are unaware of any past work using knitting as a fabrication method to produce passive RFID technology. It is desired to combine these two technologies to provide a low-cost solution for detecting information in a textile-based strain sensor. A mechanically stretched fabric-based passive RFID will have measurable changes in induced current, resonant frequency, radiation pattern, and backscattered field.

In view of the many contemplated uses of smart garments, it is further desirable to leverage recent advances in smart fabrics, yarns and knitting technology to develop new wireless monitoring devices to reduce bulk and improve comfort in monitoring devices. In particular, it is desirable to design and manufacture a line of “smart garments” that blend modes of diagnostics and monitoring with functional technologies. These smart garments would be worn by patients to improve patient care and safety in health care institutions while reducing operating costs or by athletes and at home patients. It is further desired to develop wearable technology through the exploration of versatile design concepts and to investigate manufacturing methods that are sustainable and mass customizable. The present invention addresses these and other related needs in the art.

### SUMMARY

The invention addresses the above-mentioned needs in the art by incorporating conductive yarns into knitted fabric structures. CAD systems and knitting machines manufactured by companies such as Shima Seiki, have been adapted for the production of a variety of smart garments. This mode of fabrication offers many opportunities to design and manufacture smart garments with innovative design solutions for medical apparel, home monitoring devices, and beyond.

Smart garments, or “wearable technology,” made using the techniques of the invention are made of electronic textiles using novel knitting techniques for the full integration of electronics, production methods, power, communication systems, and circuitry. However, the “smart” yarns composing the smart fabrics themselves are generally more difficult to work with than conventional yarns and thus present new design challenges. In exemplary embodiments of the invention, theses design challenges are addressed by creating electrical and mechanical structures or garments using conductive yarns in knit structures to achieve a wide variety of sensing, communication, and tactile interaction applications. Knitting machines are used to intermesh conductive yarns into loops resulting in fabrics. The knitting machine is adapted to import different types of yarns (conductive and non-conductive) directly into the knit structure. Combining conductive yarns and knitting systems in accordance with the invention allow for the integration of electrical or mechanical component designs into existing clothing fabrication processes, avoiding current limitations of attaching or gluing conductive fabrics or other components over various materials.

In exemplary embodiments, smart garments are made from flexible conductive yarns and are adapted for receiving, processing, and/or transmitting data gathered from hospital or at home patients, athletes and beyond. The garment is made from flexible conductive yarns knitted into predetermined designs corresponding to sensors and/or antennas integrated into the garment so as to receive, process, and/or transmit data gathered from the person in an active or passive manner. For example, the predetermined designs may form RFID antennas for transmitting data, RFID tags, sensors, and other electronic structures.

In exemplary embodiments, the flexible conductive yarns are knitted into a Bellyband that surrounds a uterus and is adapted to monitor uterine activity and assess fetal well-being and/or to wireless transmit the acquired data to a remote monitoring device; a onesie to be worn by a baby and including sensors integrated therein for monitoring breathing of the baby and transmitting captured data wirelessly to a remote monitor for monitoring of sudden infant death syndrome (SIDS); clothing worn during exercise by a user, the clothing including sensors integrated therein for monitoring vital signs of the user and for transmitting captured data wirelessly to a portable device data display; clothing worn during sleep by a user, the clothing including sensors integrated therein for collecting heart, lung, muscle and/or brain data in sleep studies and for transmitting captured data wirelessly to a monitoring device; or any of a number of other garments adapted to collect, process, and/or transmit data collected from a person.

The invention also includes a method of making a garment with sensors and/or antennas integrated into the garment for receiving, processing, and/or transmitting data gathered from the person in an active or passive manner. The method includes providing a planar design of a garment having one or more antenna, RFID tags, or other electronic structures to a computer aided design (CAD) knitting program, exporting a CAD specification of the garment to a knitting machine, and knitting the garment using flexible conductive fabrics to make at least the one or more antenna, RFID tags, or other electronic structures in accordance with the CAD specification. Through studies and tests of the radio frequency characteristics of different knitted fabric prototypes, the inventors have found the best knitting technique to preserve the electrical performance of the conductive yarns and to meet wireless communications and RFID standards.

### BRIEF DESCRIPTION OF THE DRAWINGS

The foregoing and other beneficial features and advantages of the invention will become apparent from the following detailed description in connection with the attached figures, of which:

FIG. 1 shows a close-up of the knitting needles and resultant fabric loops formed using such a knitting machine.

FIG. 2 illustrates conductive knitted fabric of specific stitches using the knitting machine with conductive yarns as in FIG. 1.

FIG. 3 illustrates two possible conductive fabric designs made using the techniques of the invention.

FIG. 4 illustrates the knitted conductive fabric made using the knitting machine of FIG. 1, showing the knit structure and the deformation of a rectangular shape by the introduction of different loop structure.

FIG. 5 illustrates how electrical components are knitted directly into fabric in accordance with the invention.

FIG. 6 illustrates maternity telemetry to monitor uterine activity and assess fetal well-being using conventional technology on the left with its cumbersome leads and electrodes and the “Bellyband” technology on the right made using the knitted fabric technology of the invention where the electrodes and leads are knitted into the fabric by including the conductive yarns in the knitting patterns.

FIG. 7 illustrates a Bellyband device with passive/active sensor at (a), a receiver device at (b), and a display monitoring device at (c).

FIG. 8 illustrates an embodiment of the Bellyband device with an inductive RFID chip embedded therein.

FIG. 9 illustrates a close-up view of a fully insulated and washable conductive yarn for sensing, data transmission and wearable electronics that is used by the knitting machine to construct the sensors and circuits on the Bellyband in an exemplary embodiment.

FIG. 10 illustrates sample microstrip radio frequency wireless antenna components designed, fabricated, and tested by the inventors.

FIG. 11 illustrates a sample fully-knitted dipole antenna knitted using conductive yarn.

FIG. 12 illustrates the textile antenna of FIG. 11 based on a meander line dipole layout in which each one of the two elements has folded sections.

FIG. 13 shows a comparison between the simulated and measured return loss under different levels of elongation of the antenna of FIG. 11.

FIG. 14 illustrates two plots that show azimuth and elevation planes of the radiated beam under different levels of antenna elongation of the antenna of FIG. 11.

FIG. 15 illustrates a textile RFID tag formed using a fabric dipole antenna of the type shown in FIG. 11 and RFID chip connected to the antenna, as well as the corresponding back-scatter power measurements as a result of relaxing and elongating the RFID tag to resemble respiration/contraction movements of the type experienced by the Bellyband of FIGS. 6-8.

FIG. 16 illustrates an active fabric antenna architecture wherein a low power Bluetooth module is used to measure contraction/elongation coming from a fabric-based strain gauge. The processed data is then sent to a computer or smart phone for visualization.

FIG. 17 illustrates an RFID module that incorporates a standard integrated circuit that may be used with the fabric dipole antennas of the type described herein for a passive antenna architecture.

FIG. 18 illustrates the RFID chip of FIG. 17 inductively coupled to knitted antenna arms formed in the fabric in an exemplary embodiment.

FIG. 19 illustrates the layout of a folded dipole antenna used within the RFID frequency bandwidth of 860-960 MHz for use with the Bellyband described above with respect to FIGS. 6-8.

FIG. 20 illustrates the antenna's input impedance with respect to the microchip impedance for the folded dipole antenna of FIG. 19.

FIG. 21 illustrates at (a) a wearable sudden infant death syndrome (SIDS) monitor and at (b) a remote monitoring transceiver unit, whereby an active transceiver structure conveying this mechanically-obtained respiration information may be used to transmit information to a separate unit located near the child to process and relay information pertaining to SIDS detection.

FIG. 22 illustrates a sample wearable active/passive device at (a) and a portable device data display at (b).

FIG. 23 illustrates an application in which the knitted fabrics of the invention are used to create a wireless signaling system for collecting heart, lung, muscle and brain signals in sleep studies, whereby conventional monitoring technology is shown on the left hand side of FIG. 23 while the smart fabric technology of the invention is shown on the right hand side.

FIG. 24 illustrates a bullet proof vest made with piezoelectric pressure sensors that react by applying outward pressure.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of any claimed invention. Similarly, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer both to methods and software for implementing such methods.

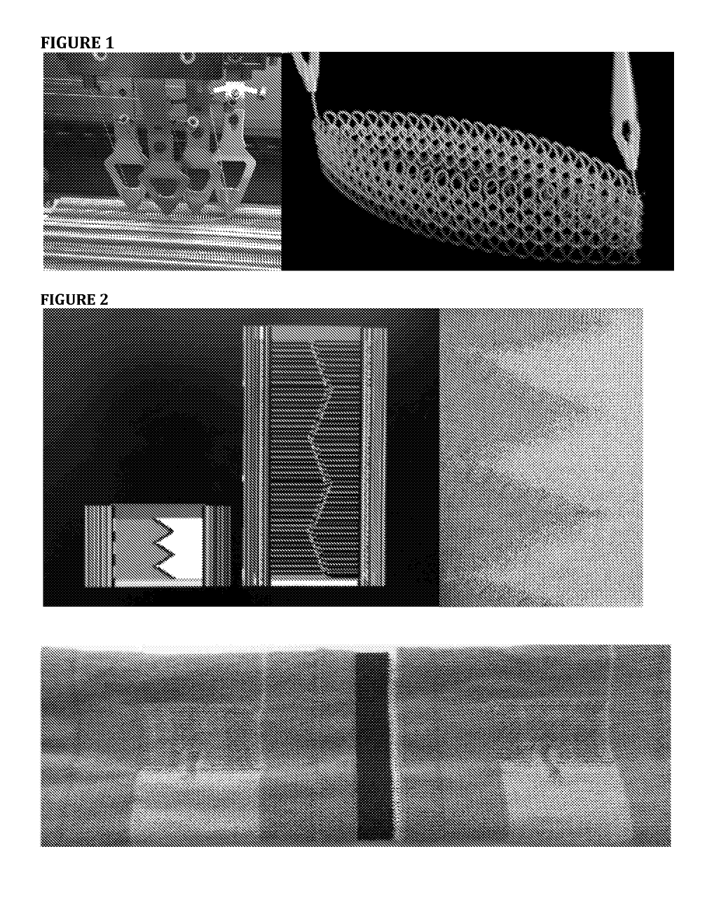

A detailed description of illustrative embodiments of the present invention will now be described with reference to FIGS. 1-24. Although this description provides a detailed example of possible implementations of the present invention, it should be noted that these details are intended to be exemplary and in no way delimit the scope of the invention. First, the knitting technology will be described, and then applications of the resulting smart garments will be described.

Active Transceiver Vs. Passive Transceiver and Mechanical Sensing Vs. Mechanical Actuation Fabric Architectures

In exemplary embodiments of the invention, conventional active and passive micro strip circuit components are realized by creating different conductive shapes using copper. Active circuits are powered, with their own energy source, to transmit, receive, and process (potentially minimally) wireless data. On the other hand, passive circuits require no external energy source, relying upon backscatter power or frequency to convey information. The knitting technology described herein is capable of realizing both active and passive transceivers using conductive threads and knitting technology.

In accordance with the invention, these active or passive transceivers can be coupled with mechanical knit structures that either actuate or sense changes in their shape to realize a rich set of applications. Mechanical sensing can be achieved by monitoring the change in conductivity, RFID backscatter frequency/power, or characteristic RF impedance of knit conductive structures as they are mechanically manipulated. Various mechanical knit structures can be achieved by infinite loop combination to facilitate mechanical actuation. A rib structure, for example, can cause a fabric with the same amount of loop to appear much smaller in one area, creating a more elastic material in that specified area. Variation in loop architecture, combined with piezoelectric or nitinol “smart” yarns as opposing forces can further provide a structure that can direct the knit structure movement to realize a rich set of applications described below.

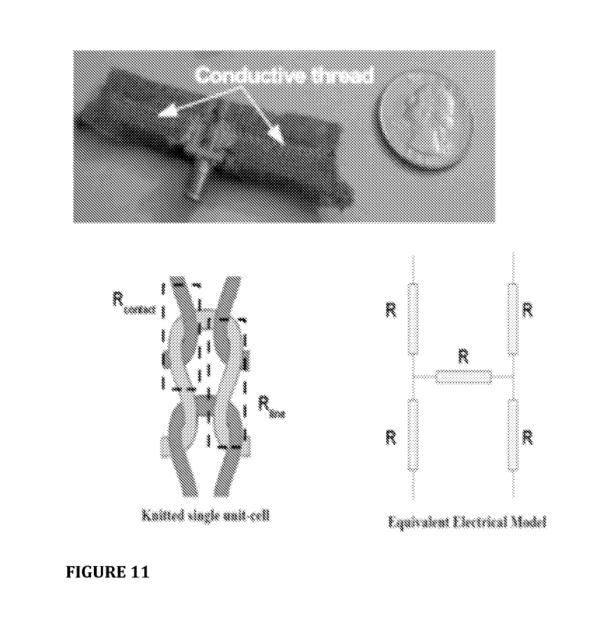

As known by those skilled in the knitting arts, knitting is the intermeshing of yarns into loops resulting in fabrics. In particular, knitting is the process of creating fabric with yarns by forming a series of interconnected loops. For electrical purposes a conductive yarn is used to produce a planar structure having finite resistivity determined by the yarn's conductivity and manufacturing method. FIG. 11 described below illustrates a knitted single unit cell that functions as a building block of the entire knitted structure.

As the contact area between the conductive loops increases, the equivalent resistance of the fabric decreases. Therefore, to ensure good conductivity of the overall design, the loops forming the antenna are tightly knitted even when the fabric is in a relaxed state. Thus, the two conductive branches of the knitted dipole antenna resemble a conventional planar design made by copper. For exemplary embodiments of the antennas discussed herein, a 99% pure silver plated nylon yarn having a linear resistance of 50Ω/M, a yield of 6400 M/Kg, a tenacity of 37 cN/tex, and an elongation of 27% was used and knitted using the Shima Seiki knitting machines described below. Those skilled in the art will appreciate that other conductive yarns may be used for different applications as appropriate.

Knits are widely used in active sportswear for their comfort and shape retention. Formfitting garments that stretch as the body moves can be constructed with specialized yarns engineered to perform specific functions. Over the last ten years, computerized knitting systems have shown great promise for wearable technology and mass customization of knitted garments. Today, advancements in specialized materials and fabrication technologies offer viable opportunities to design and knit seamless garments embedded with technology. This type of equipment could be called a rapid garment prototyping machine (like a 3D printer), with the added ability and advantage to mass-produce as needed. CRAFT, Center for Rapid Automated Fabrication Technologies at the University of Southern California, is a center with industry partners working on similar issues as they relate to buildings and objects. They aim “to develop the science and engineering needed for rapid automated fabrication of objects of various sizes up to mega-scale structures.” 3D knitted construction methods in accordance with the invention hold remarkable potential for innovative design solutions in smart textile and medical apparel.

In recognizing the potential this knitting technology presents in the production of smart garments for health monitoring and safety, the inventors have participated in a partnership with Shima Seiki, the world leader in 3D or WHOLEGARMENT™ computerized knitting. Shima Seiki's SDS-ONE APEX3™ workstation with its capability of accurately simulate fabric construction affords researchers and designers the opportunity to create and simulate prototypes, import CAD specifications of the final product, and produce made to measure or mass-produced pieces on various Shima Seiki knitting machines including the WHOLEGARMENT™ knitting machine. FIG. 1 shows a close-up of the knitting needles and resultant fabric loops formed using such a knitting machine. This type of equipment is ideal for producing prototypes fabricated with various types of yarn and stitch styles. A multiple yarn carrier system of the knitting machines enables specific placement of different types of yarns. The resulting conductive knitted fabric of specific stitches such as those shown in FIG. 2 can be innovatively used as powerful devices for intelligent uses, such as monitoring sensors and heat generators. In exemplary embodiments, the knitting machine specifically places conductive and capacitive yarns directly into garments. It can also seamlessly knit various “pockets” anywhere on the garment to hold various electronic components. Using the Shima Seiki 3D knitting machines, the inventors have been above to develop, quickly fabricate, and reconfigure samples.

In the development of the present invention, the inventors have evaluated the concept of prescription manufacture through research of knit architecture and its potential for modular and flexible production intended for a variety of medical applications. With an understanding of the capability of the advanced knitting equipment, the inventors have designed, programmed and knit a variety of fabric structures integrating a wide range of high tech yarns to develop a line of health and patient safety products. The smart garment prototypes are knitted with a combination of breathable antibacterial yarns for fit and comfort as well as insulated conductive yarns and optical fibers to accommodate the embedded technologies. These new garments have been designed to replace the current standard of bulky and cumbersome garments connected to equipment as, in many cases, such equipment is not necessarily required.

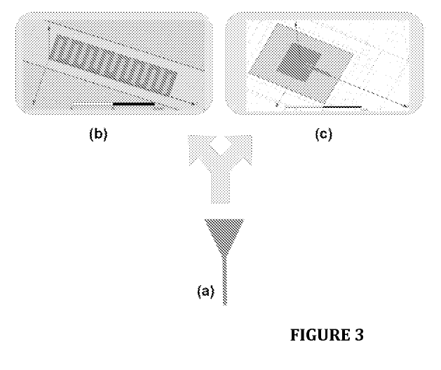

Those skilled in the knitting art will appreciate that a complete system that incorporates wearable sensors and body sensor networks within a textile will require a number of functionalities to be added to the textile structure, including conductivity, sensing, actuation, data transmission and computation. For example, FIG. 3 illustrates two possible conductive fabric designs in the form of antennae made from a knit antenna design sample knitted in a repeatable form. Data transmission is essential between components and also wireless connectivity is often desirable. As will be explained more fully below, the inventors have found that this is possible in a garment through use of flexible polymer or textile antennas.

### Prescription Manufacture Design Criteria

The inventors have developed the following general criteria to guide the prototype explorations:

Incorporate comfort, ergonomics and aesthetic in medical instrumentation;

Insure flexibility of production process through mass customization;

Use appropriate materials that can be safely sanitized for repeated wear;

Use knitting architecture to fully integrate technology into textiles; and

Design to ensure economic viability, minimize labor and waste material by leveraging the capabilities of the advanced knitting machine.

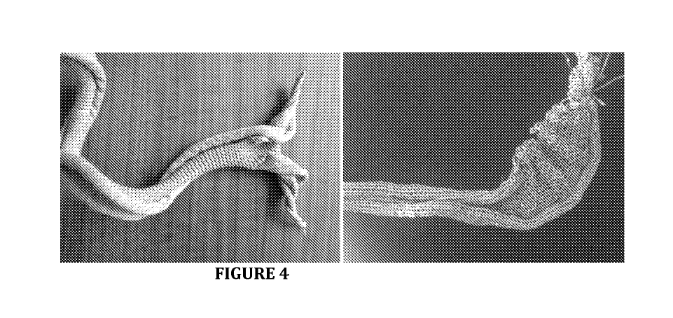

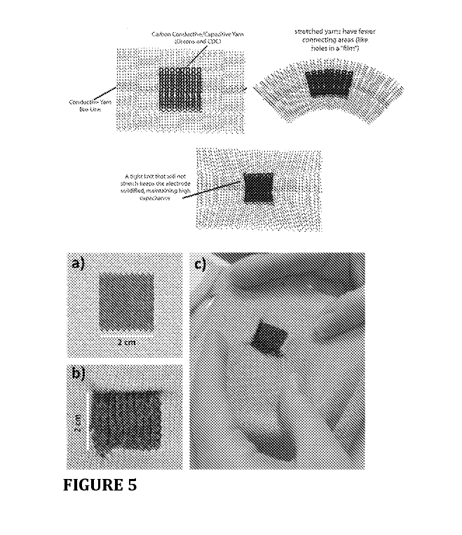

Using state-of-the-art knitting machine technology for wearable devices has several advantages over the cut and sew method. First, the implementation is directly made during the clothing fabrication process and there is no need for piecing together different materials. Additionally, knits are widely used in active sportswear for their comfort and shape retention. Formfitting garments that stretch as the body moves can be constructed with specialized yarns, engineered to perform specific functions. The resulting knitted conductive fabric such as shown in FIG. 4, which shows the knit structure and the deformation of a rectangular shape by the introduction of different loop structures, has intrinsic flexibility capable of following the movements of the human body without the need, as currently done, of copper foils glued over flexible materials to create electrical devices in wearable applications. Directly knitting electrical components into fabric as shown in FIG. 5, for example, also eliminates the use of glue or other attachment procedures that can affect the electrical characteristic of the device. The homogeneous and fully integrated knitted component has the important characteristic of being easier to fabricate by standard clothing machines, avoiding the layering process currently in use for the electro-textiles components. Stretch variations of the knitted conductive structure may be recorded as a backscatter passive RFID. When used as a Bellyband (described below), fluctuations may be detected by interrogating the RFID unit and collecting data on uterine contractions, for example. The CAD workstation with its capability of accurately simulating fabric construction affords researchers and designers the opportunity to create and simulate prototypes to be knitted seamlessly into garments embedded with technology. The CAD system enables design of specific knit structures and allows various yarns to be knitted into complex patterns corresponding to sensors and/or antenna components, for example. This type of equipment is ideal for producing prototypes fabricated with various types of yarn and stitch styles, while being fully scalable to mass production.

Designed and fabricated properly, smart garments have great potential to increase comfort and ease of movement to monitor a variety of bodily functions for patients and beyond. For example, such garments could improve efficiency for health professionals, and improve upon and replace current less efficient and bulky medical instrumentation. Smart garments can make an enormous contribution to health care by saving lives, improving care, efficiency and cost effectiveness then trickle down to home monitoring devices and many other types of monitoring. Computer aided 3D knitting is a mass customizable form of manufacturing, already in use for production in the garment industry. This mode of fabrication and manufacture, utilizing new high performance yarns and smart materials, offers a future of many opportunities for wearable technology for a variety of applications and for prescription manufacture.

### Applications

Since the conductive yarns may be used by a knitting machine to create conductive fabric structures based on inputs to an accompanying CAD system, numerous types of garments may be fabricated for numerous applications. Several such applications will be described here.

### Bellyband for Pregnant Woman Monitoring

Mechanical stretching and compression of knit fabric structures can convey vital information in the monitoring of pregnant women and other medical patients. This mechanical information can be conveyed via either active or passive knit transceiver structures. Information from other sensors designed to measure fetal and mother EKG, can also be knit and integrated into a “Bellyband” system for providing fetal monitoring.

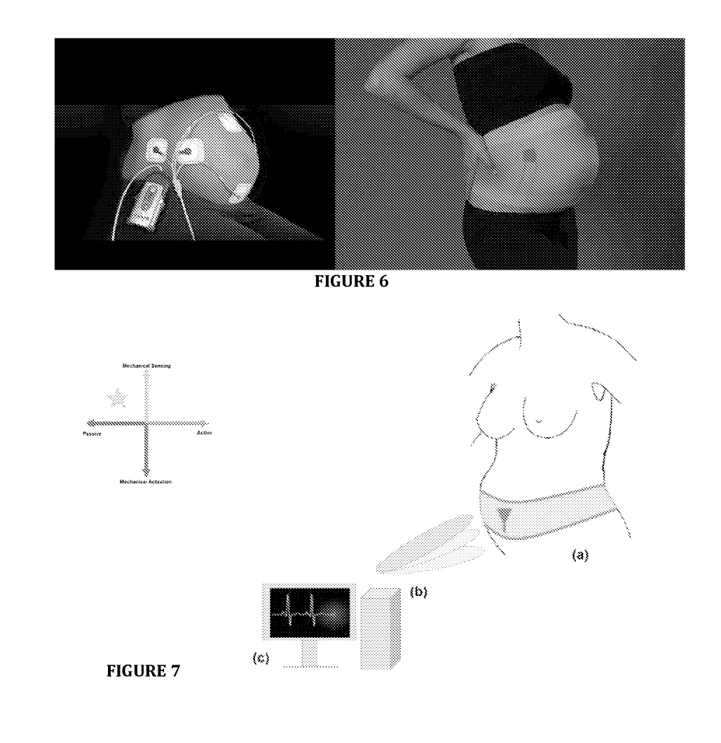

FIG. 6 illustrates a first exemplary embodiment using the smart fabric technology of the invention to provide maternity telemetry to monitor uterine activity and assess fetal well-being. On the left hand side, FIG. 6 illustrates a conventional Monica Healthcare AN24 wireless fetal monitor with its cumbersome leads and electrodes and, on the right hand side, the “Bellyband” technology made using the knitted fabric technology of the invention where the electrodes and leads are knitted into the fabric by including the conductive yarns in the knitting patterns. It will be appreciated that current portable devices, such as the Monica AN24, are still relatively cumbersome and uncomfortable with many wires attached to it. Such portable devices restrict a woman's movements and activities and do not offer true 24 hour a day, 7 days a week monitoring. FIG. 7 illustrates such a Bellyband device with passive/active sensor at (a), a receiver device at (b), and a display monitoring device at (c). The knitted smart garment known as the “Bellyband” detects and monitors uterine activity, such as fetal EKGs to assess fetal well-being, and does so wirelessly 24 hours a day, 7 days a week.

As shown in FIGS. 6 and 7, the smart fabric technology of the invention is used for monitoring uterine activity and assessing fetal well-being using a garment formed of smart fabric that is worn around the pregnant woman's uterus. Of course, it is generally known that a fetal monitor that measures a baby's heartbeat in utero in response to the contractions of the uterus is an important tool to assess fetal wellbeing. Many types of monitors exist that all serve the same function. The hospital external electronic fetal monitor standard is a two-belt ultrasound device that is strapped around the mother's belly and attached to a large box next to the labor bed, keeping the pregnant woman from moving freely and comfortably. Conventional telemetry monitoring uses radio signals to transmit the baby's heartbeat and is the newest type of monitoring available. Telemetry modeling allows expecting mothers to be monitored 24/7 while maintaining their mobility. Studies report that the use of small wearable monitoring devices for labor, delivery, and fetal monitoring that maintain the mobility of the patient is rapidly growing and medical publications support its use.

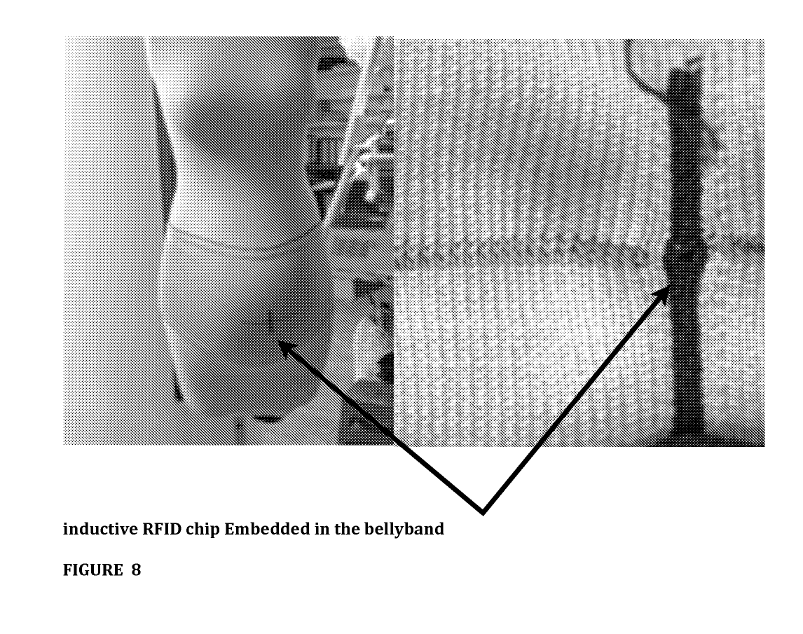

The fetal monitoring device illustrated in FIGS. 6 and 7 leverages recent advances in knitted smart fabrics and passive backscatter radio frequency identification (RFID) to develop a new wireless telemetry technology that reduces bulk, improves comfort, and enables greater mobility in pregnant women. The technology involves the creation of a washable knitted smart fabric maternity “Bellyband” to detect and monitor uterine activity as well as to pick up fetal EKGs to assess fetal wellbeing. The Bellyband is soft, comfortable, and safe while providing freedom of movement to the pregnant woman. The Bellyband also provides an improvement in current uterine monitoring standards in hospitals. As shown in FIG. 8, the Bellyband may include an inductive RFID chip embedded therein for sensing fetal activity.

In exemplary applications, the Bellyband illustrated in FIGS. 6-8 would be worn by women who are admitted to the hospital at high risk for early labor and/or fetal wellbeing. While in the “antenatal unit,” a pregnant woman wears the comfortable smart fabric Bellyband continuously allowing fetal wellbeing assessment. The high-risk pregnancy would be thus monitored safely and comfortably on a daily basis, without restricting the woman's movements and activities. 24/7 comfortable monitoring using Bellyband could help reduce millions of stillbirths per year worldwide.

As noted above, the Bellyband is made from knitted smart fabric. As an example, a Shima Seiki 3D knitting machine was programmed to knit a Bellyband to monitor contraction with the sensor yarn to become tocodynamometer. As known to those skilled in the art, a tocodynamometer (toco) is a pressure-sensitive contraction transducer that measures the tension of the maternal abdominal wall. The form-fitting Bellyband is knitted with fibers such as “coolmax” for comfort and breathability on the woman's belly and, combined with “smart yarns” for the knitted circuitry and sensors, the Bellyband is engineered to perform as a tocodynamometer. Knits are widely used in active sportswear for their comfort and shape retention and advancements in specialized materials and fabrication technologies offer viable opportunities to design and knit seamless garments embedded with technology such as the smart fabric Bellyband.

Using a Shima Seiki 3D knitting machine, a fully automated clothing construction device available to the inventors, many test samples have been developed to be quickly fabricated and reconfigured with ease. The knitting machine has the ability to import CAD specifications of clothing for fabrication with various types of yarn and stitch styles and can specifically place conductive and capacitive yarns directly into the garment at desired locations. The Shima Seiki 3D knitting machine can also seamlessly knit various “pockets” anywhere in the garment to hold various electronic components. These 3D knitted construction methods hold remarkable potential for innovative design solutions in smart garments. 3D knitting as a mass customizable form of development in high tech yarns is ideal for smart garments manufacturing. In an exemplary embodiment, yarns such as a fully insulated and washable conductive yarn for sensing, data transmission and wearable electronics is used by the knitting machine to construct the sensors and circuits on the Bellyband as shown in close-up in FIG. 9.

There are several factors used in assessing uterine activity that the Bellyband device will be able to record using sewn-in or embedded sensors. All of the following quantities can be estimated through analysis of the expansion and contractions of the knitted Bellyband:

Frequency—the amount of time between the start of one contraction to the start of the next contraction.

Duration—the amount of time from the start of a contraction to the end of the same contraction.

Intensity—a measure of how strong a contraction is. With external monitoring, this necessitates the use of palpation to determine relative strength. With an intrauterine pressure catheter (IUPC), this is determined by assessing actual pressures as graphed on a paper.

Resting Tone—a measure of how relaxed the uterus is between contractions. With external monitoring, this necessitates the use of palpation to determine relative strength. With an IUPC, this is determined by assessing actual pressures as graphed on the paper.

Interval—the amount of time between the end of one contraction to the beginning of the next contraction.

The National Institute of Child Health and Human Development (NICHD) nomenclature defines uterine activity by quantifying the number of contractions present in a 10-minute window, averaged over 30 minutes. Uterine activity may be defined as:

Normal—less than or equal to 5 contractions in 10 minutes, averaged over a 30-minute window; and

Tachysystole—more than 5 contractions in 10 minutes, averaged over a 30-minute window.

Those skilled in the art will appreciate that a wearable and portable fetal monitoring system such as the Bellyband would allow for continuous monitoring, measurement, and recording of fetal movements which have been documented to be associated with fetal well-being and reduced stillbirths. The variety of fetal movements that would be measurable after the second trimester would include, but not be limited to, spontaneous movements such as kicks, rolls, and flips. This monitoring, measurement, and recoding can be performed simultaneously with the monitoring, measurement, and recording of uterine contractions and other parameters associated with pregnancy. Such a continuous wearable and portable fetal monitoring system offered by Bellyband would reduce the potential for increased maternal anxiety associated with other methods of monitoring fetal movement and give doctors a new data set from which to evaluate the health of the baby. Bellyband will be able to count multiple types of fetal movement within specific and non-specific time periods. An alarm system may be associated with Bellyband to indicate to the data acquisition system either in real time or through archival retrieval if measured fetal movements are significantly reduced within the specified time period, e.g., less than 10 movements in 2 hours.

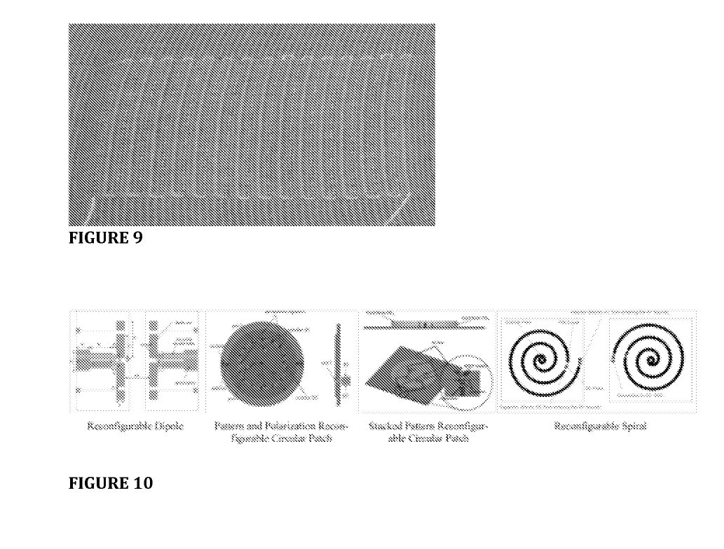

Given the sensing needs and knitting capabilities described above, there is a need to develop an integrated system to collect and process data. Microstrip antennas and transmission line circuits are ideal technologies for integration with the knitted smart fabrics. The principle behind microstrip circuits is that different circuit functionality (e.g., antennas, transmission lines, resistors, filters) can be realized by changing the shapes of a conductor. Some sample microstrip radio frequency wireless components designed, fabricated, and tested by the inventors are shown in FIG. 10, including from left to right a reconfigurable dipole, a pattern and polarization reconfigurable circular patch, a stacked pattern reconfigurable circular patch, and a reconfigurable spiral.

The inventors have used the shape-driven functionality of microstrip circuits to realize low complexity passive techniques to collect and transmit sensed uterine activity data with smart fabrics of the type described herein. These techniques are inspired by passive radio frequency identification (RFID) that do not require complex transmission circuitry or batteries for power, but instead convey information through backscatter. The knitted smart fabric system described herein can realize a Bellyband that conveys information through variable radio frequency backscatter from an external interrogating signal. Such backscatter RFID systems and microstrip architectures are described, for example, by Schubler, et al. in “Compact microstrip patch antennas for passive RFID backscatter tags,” Microwave Conference, 2009, EuMC 2009, European, pp. 1101-1104, Sep. 29, 2009-Oct. 1, 2009, and by Siden, et al. in “Fetal and maternal heart rate confusion during intra-partum monitoring: comparison of trans-abdominal fetal ECG and Doppler telemetry,” Paper presented at the Meeting of the Society for Maternal-Fetal Medicine (SMFM), California, USA, The 31^{st} Annual Meeting, February, 2011. The various components of uterine data described above will cause variations in the shape of the knitted backscatter passive RFID architecture. These fluctuations will be detected by the interrogating RFID unit. The uterine quantities described above will be extracted via signal processing performed on the signal received by the RFID interrogator as shown above in FIG. 7.

Once proven to be safe, reliable, and comfortable, the Bellyband could first be prescribed by a doctor and ultimately sold directly to pregnant woman while at home. This type of monitoring would theoretically allow intervention in case of emergency before the point of no return is reached and could help prevent premature deliveries, reduce hospital costs, and emergency procedures and could help to reduce stillbirth numbered at millions per year worldwide.

### Active and Passive Antenna Designs

FIG. 11 illustrates a sample fully-knitted dipole antenna knitted using conductive yarn. In an exemplary embodiment, the dipole antenna of FIG. 11 is a high frequency dipole antenna fabricated using conductive yarns of the type described above that are knitted using the knitting techniques described above. In the exemplary embodiment, a half-wavelength dipole antenna is provided that is centered at 868 MHz. Return loss and measured radiation patterns under different levels of fabric elongation were measured as illustrated in FIG. 13. As illustrated in FIG. 13, even under different levels of fabric elongation, the antenna shows good agreement with simulations in terms of input impedance and scattering parameters. As shown in FIG. 11, the fabric may be weaved to form knitted single unit cells that form an equivalent resistive circuit having measurable values. The radiated field resembles the predicted pattern even though small gain reduction was experienced due to the lossy nature of the conductive yarns relative to conductive metal antennas. Overall, the results demonstrate proof of principle for knitted textile-based antennas and demonstrate potential passive RFID application. The results also show that such a flexible antenna can be easily manufactured using textile fabrication methods and applied to create passive RFID-based strain gauges for use in devices such as the Bellyband described herein.

As illustrated in FIG. 11, the fabric dipole antenna may be modified by mixing more elastic and more conductive yarns to improve efficiency and gain even under elongation. Such materials permit the design of active and passive coplanar antenna architectures for Bellyband and other applications. The election of the operating frequency range of such a fabric dipole antenna is based on the stretching sensitivity and the medical/RFID frequency bands available for use.

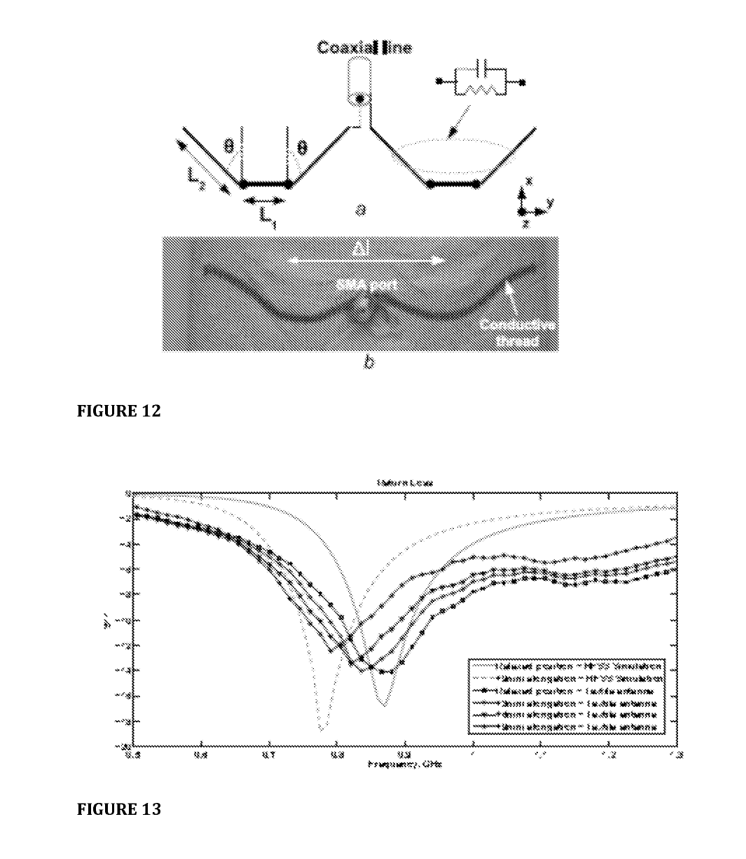

The textile antenna of FIG. 11 is based on a meander line dipole layout in which each one of the two elements has folded sections as illustrated in FIG. 12. In exemplary embodiments, each of these quarter-wavelength (AA) branches has two symmetric diagonal sections having length L₂=36 mm and one horizontal section of length L₁=14.7 mm. The overall length of the half-wavelength (λ/2) dipole is about 173 mm in the exemplary embodiment.

With respect to a straight geometry, the parasitic effect between each folded section allows for higher input impedance sensitivity in response to applied elongation. When the antenna is subjected to an external force, the shape of the antenna changes. The overall length is extended and the distance between the folded sections θ is larger as well. Thus, the partial folding of the elements is useful in reducing the antenna size, but it also significantly impacts the antenna input impedance for strain gauge applications as the distance between the folded sections changes the parasitic capacitance.

After defining the dimensions of the antenna, the design was simulated using the full-wave electromagnetic field simulator HFSS™ and tuned for a center frequency of 868 MHz corresponding to commercial passive RFID chips. As illustrated in FIG. 12 at (b), the non-conductive knitted fabric (lighter trace) supports the conductive pattern of the antenna (darker trace), and an SMA feed port was connected to the two branches of the antenna through solidified conductive paint having resistivity less than 60 mΩ/sq/mil.

The impedance integrity between the antenna port and the feed line was determined by measuring the return loss, or S₁₁, using an Agilent N5230A network analyzer. FIG. 13 shows a comparison between the simulated and measured return loss under different levels of elongation. The antenna design was first simulated in relaxed position, and was subsequently simulated by applying Δ1=9 mm of incremental length and Δθ=20° of incremental separation between the folded sections. In both cases, the resonance of the simulated antenna is in good agreement with the measured textile prototype. Once the fabric is stretched through an axial force, the S₁₁ peak starts to deviate from the designed center frequency. As the applied force increased, the length of the textile antenna increases as well, shifting the resonant frequency to lower levels. The 10 dB return loss bandwidth of the simulated dipole is about 80 MHz, while the textile antenna prototype exhibits a bandwidth of around 120 MHz. This larger value is potentially due to the more lossy nature of the fabric based antenna with respect to the simulated copper design.

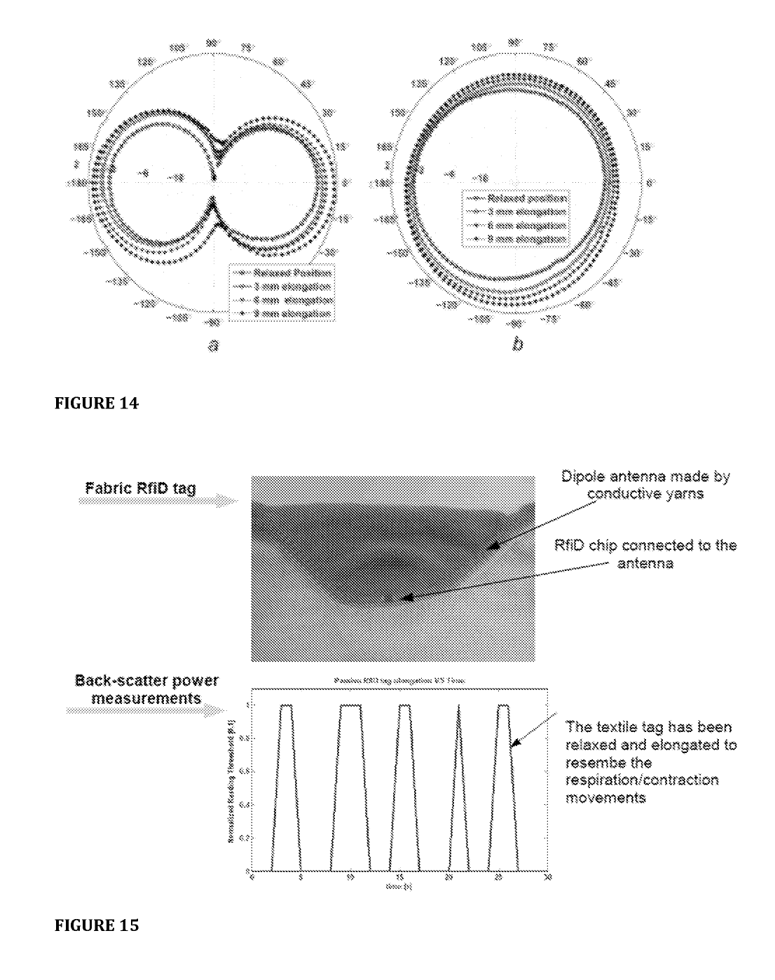

The radiation characteristics of the textile antenna have been measured within an anechoic chamber facility. Two plots in FIG. 14 show azimuth and elevation planes of the radiated beam under different levels of antenna elongation. In both relaxation and elongation conditions, the patterns resemble the typical radiation of a Hertzian dipole. Thus, the current along the textile antenna undergoes the same distribution that is established in an ideal copper based design. As in the case of return loss, the applied axial force changes the antenna length, distributed load, and gain. The total measured gain in the case of relaxed position and maximum elongation (9 mm) is respectively, −0.8 dB and 1.5 dB. The elongation of the conductive fabric causes a tighter configuration of the loops made by yarns. Consequently, the reduced sensitivity of the overall structure will lead to enhancement of the antenna gain. Due to the more lossy nature of the textile antenna, the maximum measured gain experiences a reduction of 1 dB with respect to the simulated value. However, the overall performance makes the antenna a potential candidate for strain sensor applications through the application of a passive RFID chip, described below.

FIG. 15 illustrates a textile RFID tag formed using a fabric dipole antenna of the type shown in FIG. 11 and RFID chip connected to the antenna, as well as the corresponding back-scatter power measurements as a result of relaxing and elongating the RFID tag to resemble respiration/contraction movements of the type experienced by the Bellyband of FIGS. 6-8.

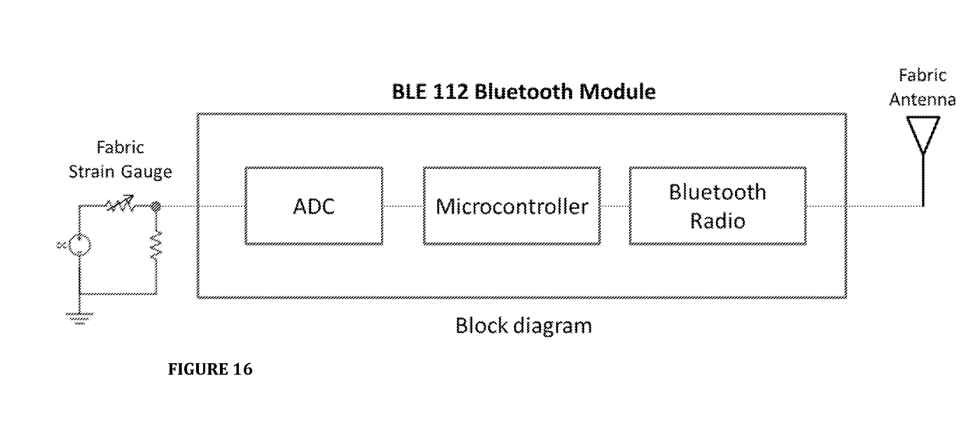

FIG. 16 illustrates an active fabric antenna architecture wherein a low power Bluetooth module 200 is used to measure contraction/elongation of a fabric-based strain gauge 202. The data in then processed and sent to a computer or smart phone for visualization. Such a Bluetooth module 200 preferably has a very small dimension on the order of 10 mm×20 mm and very low power consumption in the μA range. As illustrated, ADCs 204 may be used for analog to digital conversions and a microcontroller 206 made be used to process the resulting digital signals for transmission via Bluetooth radio transceiver 208 to the fabric antenna 210 described herein. The Bluetooth module 200 is located in an exemplary embodiment on the stomach portion of the Bellyband to measure resistive variations during contractions/elongations and to capture such measurements in the Bluetooth module 200. The data is processed and sent to, for example, a smartphone device via Bluetooth module 200 and fabric antenna 210. A customized app running on the smartphone device may be used to visualize the data locally.

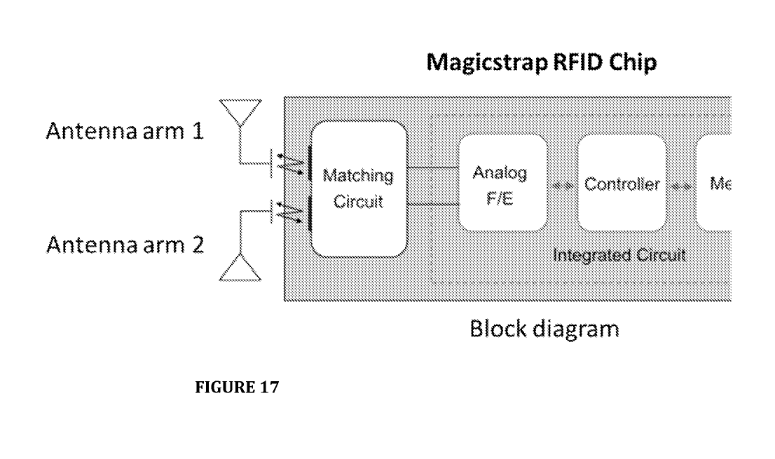

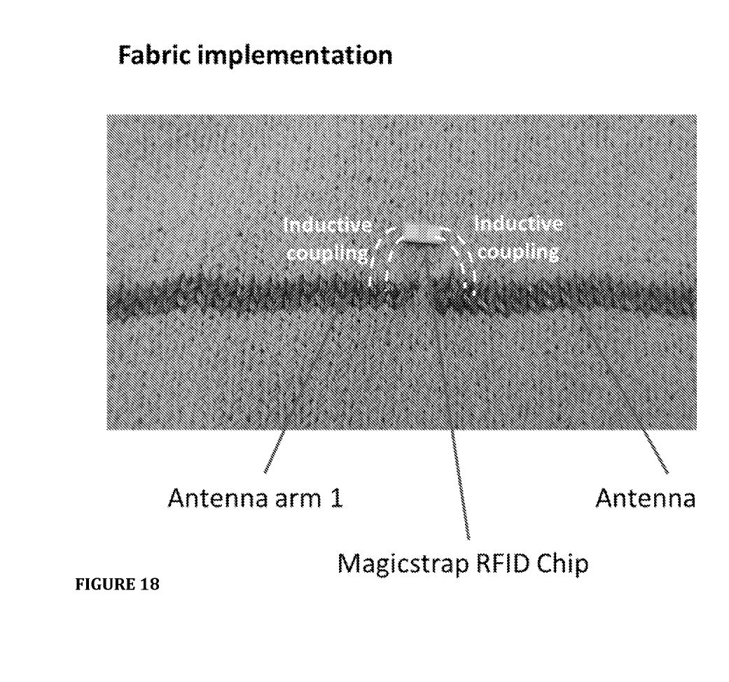

FIG. 17 illustrates an RFID module that incorporates a standard integrated circuit that may be used with the fabric dipole antennas of the type described herein for a passive antenna architecture. As opposed to standard RFID chips, such an RFID module does not need to be soldered to the antenna design as the radio-frequency signal is inductively coupled to the antenna. FIG. 18 illustrates the RFID chip of FIG. 17 inductively coupled to knitted antenna arms 212 formed in the fabric in an exemplary embodiment. As with the antennas described above, elongation/contraction of the fabric antenna produces a change in the measured backscatter power levels that may be detected by an RFID reader. In the Bellyband embodiment as shown in FIG. 8, the RFID chip 100 is placed, for example, on the stomach portion of the Bellyband for detection of elongation/contraction of the Bellyband fabric antenna and transmission of the changed backscatter power levels to a nearby RFID reader. Inductive coupling of the RFID chip 100 to the fabric antenna has been shown to provide greater sensitivity to the elongation of the fabric antenna though more dramatic fluctuations in received signal strength indicators. The greater signal strength variations are caused by both the change in radiation characteristics of the antenna as the fabric stretches as well as inductive decoupling of the RFID chip from the antenna as the fabric stretches.

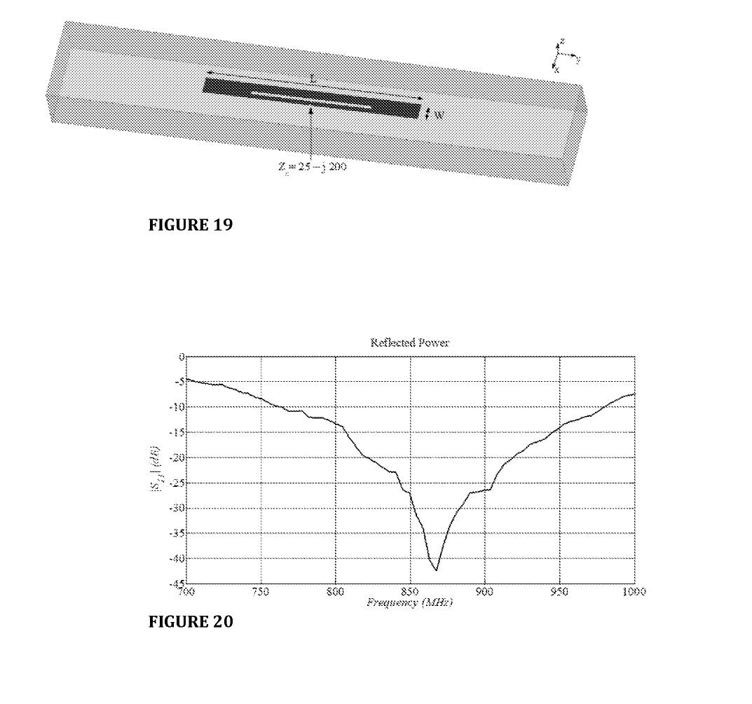

The RFID microchip 300 shown in FIG. 17, referred to as the MAGICSTRAP™, can enhance the sensitivity on a wireless strain sensor while maintaining the full flexibility of the fabric design. The MAGICSTRAP™ microchip 300 requires a specific antenna input impedance in order to operate at the maximum performance. As illustrated, the MAGICSTRAP™ microchip 300 includes a matching circuit 302 that matches the received signals from the knitted antenna arms 212 and provides the results to memory 304 via analog to digital converter 306 and controller 308. To maximize use of the MAGICSTRAP™ microchip 300, another design of the Bellyband antenna is proposed. As shown in FIG. 19, another embodiment of the Bellyband antenna includes a knitted folded dipole antenna, specifically designed for the use within the RFID frequency bandwidth 860-960 MHz. In order to validate the manufactured prototype, the inventors conducted extensive electrical characterization with a vector network analyzer and the results show good complex conjugate impedance matching with the microchip input ports.

By using the RFID microchips 100 or 300 described herein, the physical deformation of the RFID tag causes impedance variations and coupling reduction between the microchip and the antenna, yielding significant variations of the backscattered power (RSSI). For these reasons, the inventors have selected the RFID microchip Murata MAGICSTRAP™ LXMS31ACNA-011, a small 3.2×1.6 mm SMD microchip with input impedance equal to Zc=25−j 200 between 870 and 915 MHz. Due to the negative imaginary part of the microchip impedance Zc, the antenna was designed to exhibit inductive reactance for complex conjugate matching (Za=25+j 200) and maximum energy delivery from the microchip. FIG. 19 illustrates the 3D model of the antenna. The layout has been designed and simulated using the High Frequency Structure Simulator HFSS. The design is made by a folded dipole, with a thin long slot which plays the role of tuning the center frequency of the impedance matching. The outer dimensions of the antenna are W=9.5 mm and L=124 mm, while the internal slot length is 77 mm and width equal to 3 mm. The gap between the two dipole arms has been designed having length of 1 mm considering the distance between the RFID microchip pads.

Once the antenna of FIG. 19 was properly tuned to approach the ideal complex conjugate matching at 870 MHz, the reflection coefficient S11 between the antenna and the microchip has been measured and is shown in FIG. 20. FIG. 20 illustrates the input impedance of the antenna of FIG. 19 with respect to the microchip impedance of the microchip of FIG. 17. As illustrated in FIG. 20, the peak is well below −10 dB at the expected resonant frequency, proving the good impedance matching with the MAGICSTRAP™ microchip.

As a result of pairing the microchip of FIG. 17 with the antenna of FIG. 19, the reading range of the Bellyband has been significantly increased, as well as the sensitivity to contraction/elongation. This antenna configuration may also be used for radial strain measurements within an anechoic chamber facility. An experimental setup comprised the manufactured tag antenna placed 1.6 m apart from a commercial Impinj Speedway RFID reader. The antenna was progressively bent from the rest position θ=0° to the maximum angle of θ=30°. For each angle, the reader interrogated the tag antenna collecting a total of 300 samples of backscattered RSSI values, recorded for following statistical analysis. With respect to the rest position, the difference in backscattered power was appreciable for angles above θ=10° and the maximum RSSI variation was of 20% at θ=26°. FIG. 19 depicts the antenna design.

### Antennas and Transceivers for Wireless Devices (e.g., RFID, Cell Phones, Local Area Networking Products)

The highly pervasive nature of wireless communication devices (e.g., RFID, cell phones, local area networking products) coupled with knitted fabric electronic technology has the potential to realize wearable wireless network transceivers. As desired, the Bellyband or other wearable device may interface with a smartphone via knitted antennas to communicate with a doctor or provide local remote processing using software applications on the smartphone.

### Early Warning System for Sudden Infant Death Syndrome (SIDS)

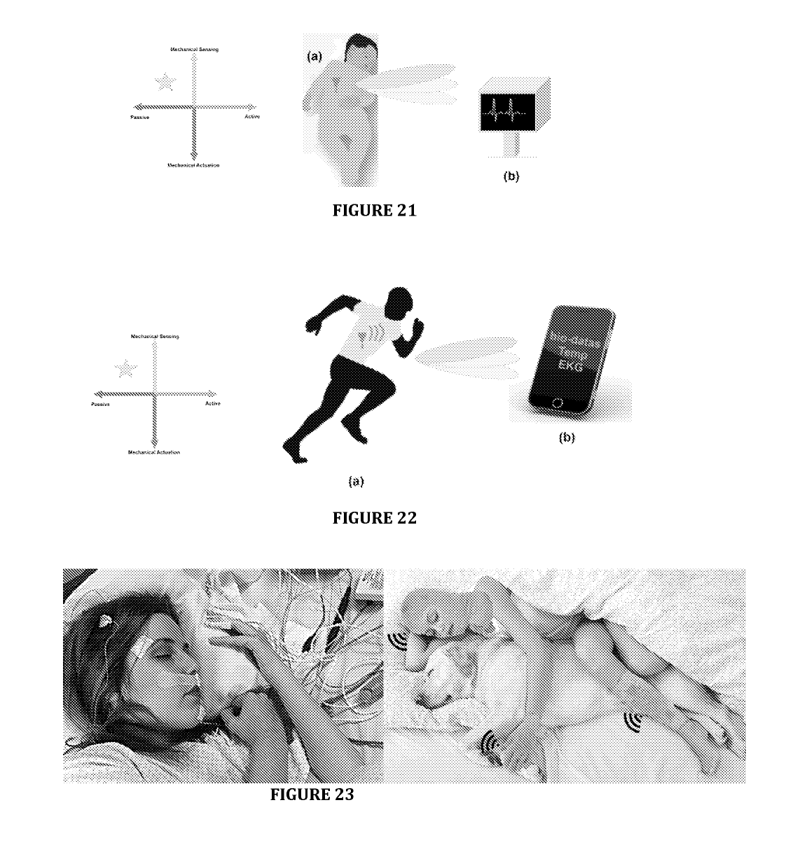

Mechanical stretching and compression of knit fabric structures can convey vital information in the monitoring of respiration in “onesies” worn by babies. As shown in FIG. 21, which illustrates at (a) a wearable sudden infant death syndrome (SIDS) monitor and at (b) a remote monitoring transceiver unit, an active transceiver structure conveying this mechanically-obtained respiration information may be used to transmit information to a separate unit located near the child to process and relay information pertaining to SIDS detection. A passive transceiver can also be used, wherein a separate unit located near the child transmits a signal to the passive knit transceiver structure. Then, in a mode of operation similar to passive RFID systems, information regarding the mechanically-obtained respiration information can be inferred by the backscatter power or frequency. In the active transceiver implementation of this technology, an energy source (e.g., a battery) would need to be integrated into the babies' clothing whereas the passive transceiver system could conceivably be a completely knit system.

### Athlete Monitoring System

Mechanical stretching and compression of knit fabric structures can also convey respiration information for athletes as part of a bio-feedback system. FIG. 22 illustrates a sample wearable active/passive device at (a) and a portable device data display at (b). In such an embodiment, information can be conveyed in either an active or passive fashion as described above for other applications. The information can be processed by a smartphone or other portable wireless device worn by the athlete to monitor and provide feedback to the athlete as they exert themselves. The incorporation of other knit sensor structures (e.g., temperature, EKG) has the potential to greatly expand the functionality of this system to unobtrusively monitor athletes to optimize their performance or warn them in the event of danger.

### Massage and Stimulation System

For people with medical conditions that limit their mobility, bedsores are a serious problem that is difficult to treat. The active and passive monitoring of mechanical stretching in the clothing of these patients can provide an indication and early warning system that their position needs to be changed. Furthermore, by applying the mechanical actuation properties of smart fabrics, an active massage and stimulation system can also be integrated into the clothing to promote healing and/or prevention of bedsores.

### Mood Fabric

Using knit sensor systems in clothing that respond to chemical, temperature, and humidity conditions allows for the development of “mood fabric” that can adapt itself in some way to the measured “mood” of the person. While not necessarily a particularly accurate way of determining the thoughts or emotions of the user, this system may also have fashion or entertainment value.

### Smart Yoga

The ability to monitor respiration, temperature, and heart rate in an unobtrusive fashion using knit sensors can have potential sports and recreational value in the development of “smart yoga” outfits. These outfits would unobtrusively monitor the state of their wearers and provide valuable bio-feedback information for improving yoga performance.

### Child Tracking and Localization

Active and passive fabric based transceivers can be used to develop RFID tracking systems for integration in the clothing of children. While there are currently RFID and GPS based systems that track children, the ability of our smart fabric techniques to seamlessly integrate into a host material provides the potential for a completely unobtrusive system. Unique identifying information can be incorporated into the active or passive RFID, and this children's clothing can be effectively networked from a smartphone or local area network application controlled by the children's parents.

### Wearable E-Commerce

There is increased interest in using RFID and near field communication based systems for electronic commerce applications. Rather than have these tags reside in a mobile device, or in a credit card, the use of smart textiles allows for the possibility of incorporating this functionality into the clothing of everyday users.

Nursing Home and/or Sleep Monitoring System

The biometric sensing and localization functionality described above can be used for monitoring the health and whereabouts of nursing home residents. In addition, as shown in FIG. 23, the knitted fabrics of the invention may be used to create a wireless signaling system for collecting heart, lung, muscle and brain signals in sleep studies. Conventional monitoring technology is shown on the left hand side of FIG. 23 while the smart fabric technology of the invention is shown on the right hand side. As shown, the monitoring wires are eliminated by the invention.

### Internet of Things

In the future, technologies like IPv6 that have a very large address space will allow for the “internet of things” in which virtually all human made objects can be networked together. The smart fabric technology of the invention may be used to encode IPv6 addresses in fabric. Furthermore, the active and passive fabric based transceivers of the invention can be used to transmit and receive information to these devices. When coupled with the biometric monitoring, localization, and mechanical actuation capabilities of smart fabrics, the potential next generation internet applications of this technology are enormous. For example, the clothing could inform the user, through active or passive transceiver methods, as to when it is worn out and needs to be replaced.

### Knit Clothing Anti-Theft Detection Tags

Electronic theft tags currently implemented using external tags or devices can be implemented using active or passive fabric transceivers. This technology would allow for tracking of stolen goods not only around the store, but also more easily at greater distances than conventional techniques.

### Smart Bulletproof Vest

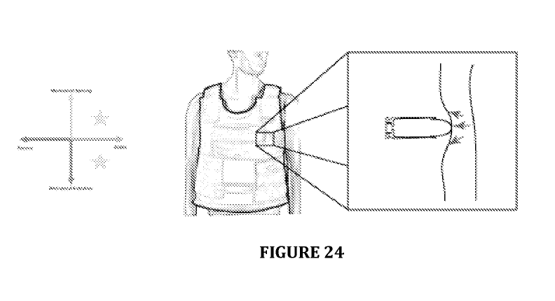

Knit structures can be made not only out of conductive threads, but potentially bullet resistant materials as well. These materials could have the ability to use piezoelectric energy harvesting and mechanical actuation to develop “reactive” armor for bulletproof vest applications. FIG. 24 illustrates a bullet proof vest made with piezoelectric pressure sensors that react by applying outward pressure.

### Automatic Resizable Clothing (or Active Skinny Jeans)

The sensing and mechanical actuation properties of knit structures allows for the development of automatic resizable clothing. Thus, clothing could be made that adapts itself to the size, shape, and condition of the person wearing the clothing.

### Tactile Telepresence (or Reach Out and Touch Someone)

Virtual and augmented reality systems are increasingly using tactile feedback to convey information from the virtual world to a user. Smart clothing with tactile sensors and actuators can be implemented for users to have more interactive experiences in virtual worlds.

### RFID Tags

A purely textile antenna associated with novel inductively-coupled RFID chips as described above can be easily integrated into clothing and other fabrics for at least the following applications:

Smart Laundry:

As part of conventional clothing manufacturing, a small RFID antenna can be knitted and equipped with an RFID chip containing the digital ID relative to the fabric characteristics such as color and fabric material. The washing machine will be equipped with an RFID reader, along with either standard or reconfigurable antennas, in order to read the garments that are inserted. As a result, each garment will be detected and a proper algorithm, running on the reader, will recognize possible conflicts of colors or fabrics in order to prevent errors of the laundry process. The reader will also enable tracking of the laundry from washer to dryer so that all laundry is accounted for. This solution can be classified as a purely passive sensing design.

Industrial Laundry Improvement:

The system described above can be used for the improvement of industrial laundries process. The manual selection of the different fabrics can be replaced with the above-described RFID solution whereby each fabric is manufactured with a knitted antenna along with RFID chip. Before entering the washing machines, an automatic chain process populated with RFID readers will select and route the fabrics based on the digital information transmitted from the chips and received from the system. The RFID readers may also be used to track the clothing during the laundering process. This solution involves a powered active RFID reader and a purely passive RFID tag.

Troop Recognition:

The knitted RFID tag, constituted by a knitted antenna along with RFID chip, can be integrated into the conventional military uniforms in order to enhance the security level of military bases. For instance, during foreign operations the gates of each base can be equipped with RFID reading system ensuring that each individual that enters the base or other facility belongs to the appropriate troop or is otherwise authorized to enter the facility. As a result, the base will enhance its level of security using low cost RFID technology. This solution also involves a powered active RFID reader and a purely passive RFID tag.

### Inductive Antenna Matching

As noted above, conventional fabric-based antennas are glued or sewed on stretchable substrates, requiring additional cost and lowering of the RF performance. However, a fully knitted structure as described herein, characterized by natural flexibility, does not restrict the user's activity. A potential drawback of such a flexible antenna is that its electrical length may be extended or compressed by the user's movements, causing changes in its characteristic impedance (or, equivalently, cause a frequency shift). In order to prevent this impedance variation when the device is bent or stretched, the inventors propose a new class of frequency reconfigurable antennas, where an adaptive matching network, in proximity of the inductive RFID chip pads, is able to prevent the impedance variations of the antenna by such types of deformation. Any distributed impedance matching network made by microstrip lines is determined by the width and length of the open or shorted stubs that constitute the reactive elements. Similarly, the dimensions of a fabric-based matching network can be reconfigured by the bending or stretching of the garment where it is integrated. Consequently, by adding this adaptive matching network to a fabric-based antenna, it is possible to prevent any impedance variation of the RFID tag, and thus improve the stability of the overall RFID system. This solution can be considered as a passive actuation, as it performs a radio-frequency adaptation using a passive knitted matching network.

### Characterizations of Different Applications for Smart Fabrics

The different applications for smart fabrics as described herein can be described based on their positions along 3 different axes: sensing versus actuating, passive versus active, and powered versus unpowered. These different axes may be defined as follows:

Sensing:

Sensing and detecting body movements and posture can be done by taking advantage of the change in the electrical or radio-frequency characteristics of a smart fabric. For example, by using a textile antenna, equipped with a passive RFID microchip, the physical deformation of the conductive layout will produce a variation of the backscattered power (RSSI) received from an RFID interrogator. The physical deformation produces a change in the antenna's resonant frequency and decoupling from the RFID microchip that can be detected as variation of the backscattered power seen from the RFID reader.

Actuation:

Garments can be adapted to contain electric wires integrated into the fabric that can change the shape of the garment itself

Passive:

A passive solution takes advantage of the change in the electrical or radio-frequency characteristics of a textile sensor. For instance, passive sensing can be achieved through the change in the electric resistance of a textile-based strain gauge, or from the change in the input impedance or radiation pattern of a textile antenna.

Active:

On the other hand, active sensing defines the ability to knit commercial off-the-shelf devices on smart fabrics. These devices are typically small powered microchips or modules. These powered devices can be sensors for biomedical monitoring, small Wi-Fi or Bluetooth modules, etc. As opposed to the passive solution, in this case powered sensors are used instead of unpowered sensors.

Powered:

Powered is any smart textile solution that requires external power source such as a battery or power supply.

Unpowered:

Unpowered smart textiles do not require any power connection to battery or power supply. Unpowered solutions can take advantage of passive sensors (textile antenna sensors, resistive strain gauge, etc.). If DC energy is needed, the smart textile can integrate a power harvesting system. For example, DC energy can be harvested from the surrounding wireless networks or cell phone networks through a wearable power harvesting system, and this system can store the harvested energy inside a textile supercapacitor for charging smart sensors of modules integrated into the garment.

The sample applications described herein can be placed along these axes as follows:

Classification of the applications:

| Application | Axis |
|---|---|
| Antennas and Transceivers for | Active + Powered |
| Wireless Devices | |
| SIDS Monitor | Passive (or Active) + Sensing |
| Athlete Monitoring System | Passive (or Active) + Sensing |
| Wearable Power Harvesting System | Passive + Unpowered |
| Massage and Stimulation System | Passive + Sensing (for |
| | monitoring) − Active + Actuation |
| | (for treatment) |
| Mood Fabric | Passive (or Active) + Sensing |
| Smart Yoga | Passive (or Active) + Sensing |
| Child Tracking and Localization | Passive (or Active) + Sensing |
| Wearable E-Commerce | Passive + Sensing |
| Nursing Home and/or Sleep | Active + Powered |
| Monitoring System | |
| Internet of Things | Passive (or Active) + Sensing |
| Knit Clothing Anti-Theft | Passive (or Active) + Actuation |
| Detection Tag | |
| Smart Bulletproof vest | Passive + Actuation |
| Automatic Resizable Clothing | Passive (or Active) + Actuation |
| (or Active Skinny Jeans) | |
| Tactile Telepresence (or Reach | Passive + Sensing (for sensors) − |
| out and Touch Someone) | Active + Actuation (for actuators) |

Insubstantial changes from the claimed subject matter as viewed by a person with ordinary skill in the art, now known or later devised, are expressly contemplated as being equivalently within the scope of the claims. For example, those skilled in the art will appreciate that the knitted sensors and knitted RFID antennas described herein may be integrated into the same knitted electrical component. Therefore, obvious substitutions now or later known to one with ordinary skill in the art are defined to be within the scope of the defined elements.

What is claimed:

1. A knitted garment adapted to receive, process, and/or transmit data gathered from a wearer of the garment, comprising at least one flexible conductive yarn directly knitted into electrical components in the garment, the knitted electrical components including at least one RFID antenna integrated into the garment so as to receive, process, and transmit data in an active or passive manner to a remote monitoring or display device.

2. The garment of claim 1, wherein said at least one RFID antenna comprises a knitted dipole antenna.

3. The garment of claim 2, wherein said knitted dipole antenna has a meander line dipole layout.

4. The garment of claim 2, further comprising an RFID chip that is inductively coupled to the knitted dipole antenna.

5. The garment of claim 4, wherein said RFID chip contains a digital ID identifying fabric characteristics of said garment.

6. The garment of claim 4, wherein said RFID chip contains a digital ID identifying a wearer of the garment as authorized to enter into a facility

7. The garment of claim 1, wherein said at least one RFID antenna is knitted so as to form an RFID tag.

8. The garment of claim 1, wherein said at least one RFID antenna comprises a folded dipole antenna having a frequency in an RFID frequency bandwidth of 860-960 MHz.

9. The garment of claim 1, further comprising a Bluetooth module that measures contraction and/or elongation of the at least one flexible conductive yarn and sends the measurements to a remote smartphone or computer via said at least one RFID antenna.

10. The garment of claim 1, wherein said knitted electrical components include at least one knitted sensor for collecting data from the wearer of the garment.

11. The garment of claim 10, wherein said at least one knitted sensor and said at least one RFID antenna are integrated into the same knitted electrical component.

12. The garment of claim 1, wherein the flexible conductive yarn is knitted into a bellyband that surrounds a uterus and said at least one sensor is adapted to monitor uterine activity and/or to assess fetal well-being and said at least one RFID antenna is adapted to wirelessly transmit the acquired data to a remote monitoring device.

13. The garment of claim 1, wherein the flexible conductive yarn is knitted into a onesie to be worn by a baby, said at least one sensor being integrated therein and adapted to monitor breathing of the baby and said at least one RFID antenna adapted to transmit captured data wirelessly to a remote monitor for monitoring of sudden infant death syndrome (SIDS).

14. The garment of claim 1, wherein the flexible conductive yarn is knitted into clothing worn during exercise by a user, said at least one sensor being integrated therein and adapted to monitor vital signs of the user and said at least one RFID antenna adapted to transmit captured data wirelessly to a display of a portable device.

15. The garment of claim 1, wherein the flexible conductive yarn is knitted into clothing worn during sleep by a user, said at least one sensor being integrated therein and adapted to collect heart, lung, muscle and/or brain data during sleep and said at least one RFID antenna adapted to transmit captured data wirelessly to a monitoring device.

16. A method of making a knitted garment with sensors and/or antennas integrated into the garment for receiving, processing, and/or transmitting data gathered from a wearer of the garment in an active or passive manner, comprising the steps of:
providing a planar design of a garment having at least one sensor and/or at least one RFID antenna knitted directly therein to a computer aided design (CAD) knitting program,
exporting a CAD specification of the garment to a knitting machine, and
knitting, using said knitting machine, at least one flexible conductive yarn directly into said at least one sensor and/or said at least one RFID antenna of said garment in accordance with the CAD specification.

17. The method of claim 16, wherein said planar design comprises a design of a knitted dipole antenna.

18. The method of claim 17, wherein said planar design of said knitted dipole antenna comprises a meander line dipole layout.

19. The method of claim 16, further comprising inductively coupling an RFID chip to the knitted dipole antenna.

20. The method of claim 16, wherein said planar design comprises a pattern for a knitted RFID tag.

21. The method of claim 16, further comprising connecting a folded dipole antenna to said at least one sensor, said folded dipole antenna having a frequency in an RFID frequency bandwidth of 860-960 MHz.

22. The method of claim 16, further comprising inductively coupling a Bluetooth module to said at least one sensor, said Bluetooth module adapted to measure contraction and/or elongation of the at least one flexible conductive yarn and to send the measurements to a remote smartphone or computer.

23. A knitted garment adapted to surround a patient's uterus and to receive, process, and/or transmit data gathered from the patient, comprising at least one flexible conductive yarn directly knitted into at least one sensor adapted to monitor uterine activity and/or to assess fetal well-being and directly knitted into at least one RFID antenna adapted to wirelessly transmit the acquired data to a remote monitoring device.

24. The garment of claim 23, further comprising an RFID chip that is inductively coupled to the RFID antenna.

25. The garment of claim 23, further comprising a Bluetooth module that measures contraction and/or elongation of the at least one flexible conductive yarn and sends the measurements to said remote monitoring device.
